(19) United States
(12) Patent Application Publication
(10) Pub. No.: US 2024/0255518 A1
(43) Pub. Date: Aug. 1, 2024

(54) CHARACTERIZATION OF SERINE-LYSINE CROSS-LINK IN ANTIBODY HIGH MOLECULAR WEIGHT SPECIES

(71) Applicant: Regeneron Pharmaceuticals, Inc., Tarrytown, NY (US)

(72) Inventors: Yuetian Yan, Chappaqua, NY (US); Gaoyuan Liu, White Plains, NY (US); Shunhai Wang, Scarsdale, NY (US)

(21) Appl. No.: 18/421,538

(22) Filed: Jan. 24, 2024

Related U.S. Application Data
(60) Provisional application No. 63/442,575, filed on Feb. 1, 2023.

(51) Int. Cl. G01N 33/68; C12Q 1/37; G01N 30/02; G01N 30/06; G01N 30/16; G01N 30/72

(52) U.S. Cl. CPC G01N 33/6848; C12Q 1/37; G01N 30/16; G01N 30/7266; G01N 33/6857; G01N 2030/027; G01N 2030/067; G01N 2333/96413; G01N 2333/96416; G01N 2333/96419; G01N 2333/976

(57) ABSTRACT

The present invention generally pertains to methods of characterizing of a protein of interest. In particular, the present invention pertains to the use of post-column denaturation, size exclusion chromatography and mass spectrometry for detecting, identifying and characterizing crosslinking amino acid residues in a therapeutic antibody.

### CROSS-REFERENCE TO RELATED APPLICATIONS

This application claims the benefit of U.S. Provisional Application No. 63/442,575, filed Feb. 1, 2023, which is incorporated by reference herein in its entirety.

### FIELD

This application relates to methods for characterization of therapeutic antibodies.

### BACKGROUND

High molecular weight (HMW) species are a critical quality attribute of therapeutic proteins due to their potential impact on both drug efficacy and safety. Therapeutic proteins often contain HMW species that are generated during product manufacturing, shipping, and storage. With size ranges from soluble oligomers to visible particles, HMW species could potentially elicit unwanted immunogenic responses, compromising a drug's safety and efficacy.

The formation of HMW species is a highly complex process and may occur under different physical, chemical, and enzymatic stress conditions. Additionally, a protein's primary sequence and higher order structure may also affect its susceptibility to interact and aggregate via different mechanisms. The generated HMW species may exhibit diverse characteristics, including size, interaction nature, conformation, and modifications. Because of its complexity, characterizing and understanding the formation mechanism of HMW size variants is very challenging, and has largely been performed only at intact or subunit levels.

Therefore, there exists a need for sensitive methods and systems for characterizing HMW species of a therapeutic protein at the local residue level, including understanding the formation mechanism and crosslinking chemistry of HMW species. This disclosure sets forth novel methods and systems for characterizing HMW species or size variants of a protein of interest, including post-column denaturation, native size exclusion chromatography-mass spectrometry, denaturing size exclusion chromatography-mass spectrometry, nano reverse phase liquid chromatography-mass spectrometry, intact analysis, subunit analysis, bottom-up analysis, imine reduction, acid treatment, isotope labeling, and combinations thereof. In exemplary embodiments, the novel systems and methods described herein were used for successfully identifying and characterizing a novel cross-link formed in the production of therapeutic antibodies.

### SUMMARY

Systems and methods have been developed for characterizing covalent crosslinking of a protein of interest. In an exemplary embodiment, a sample including a therapeutic antibody and a crosslinked variant thereof may be subjected to post column denaturation (PCD)-assisted native size exclusion chromatography-mass spectrometry (nSEC-MS) analysis to characterize the covalent cross-link. The amino acid residues forming the covalent cross-link can be specifically identified using chromatographic mobile phases with varying pH, acid treatment, reduction with NaBH₃CN, isotope labeling, and/or peptide mapping. The disclosed systems and methods for characterizing and identifying covalent cross-links formed during the production of proteins of interest may be used to inform the manufacturing, formulation, storage, and analysis of therapeutic proteins.

In a specific embodiment, a method is provided for identifying a covalent cross-link in a dimer of a therapeutic antibody (mAb), including obtaining a sample including a therapeutic mAb and at least one dimer of said therapeutic mAb comprising a cross-link; subjecting said sample to size exclusion chromatography (SEC) to form at least one fraction including said at least one dimer; collecting said fraction to form an enriched high molecular weight (HMW) sample; obtaining a first mass measurement of said covalent cross-link, said obtaining comprising, (i) contacting a portion of said enriched HMW sample to PNGase F to form a deglycosylated HMW sample; (ii) subjecting said deglycosylated HMW sample to SEC under native conditions (nSEC) to form an SEC eluate; (iii) contacting said SEC eluate to a denaturing solution to form a denatured SEC eluate; (iv) subjecting said denatured SEC eluate to mass spectrometry analysis to obtain at least one mass measurement of said at least one dimer and at least one mass measurement of at least one non-covalent dimer of said therapeutic mAb, wherein said mass spectrometer is connected in-line to said SEC column of step (ii); and (v) comparing said mass measurements of step (iv) to obtain a first mass measurement of said covalent cross-link; obtaining a second mass measurement of said covalent cross-link, said obtaining comprising, (i) contacting a portion of said enriched HMW sample to PNGase F to form a deglycosylated HMW sample; (ii) contacting said deglycosylated HMW sample to IdeS to form a fragmented HMW sample; (iii) subjecting said fragmented HMW sample to partial reduction and alkylation to form a reduced fragmented HMW sample; (iv) subjecting said reduced fragmented HMW sample to nSEC to form an SEC eluate; (v) contacting said SEC eluate to a denaturing solution to form a denatured SEC eluate; (vi) subjecting said denatured SEC eluate to mass spectrometry analysis to obtain at least one mass measurement of at least one subunit dimer of said at least one dimer and at least one mass measurement of each of the component subunits of said at least one subunit dimer, wherein said mass spectrometer is connected in-line to said SEC column of step (iv); and (vii) comparing said mass measurements of step (iv) to obtain a second mass measurement of said covalent cross-link; characterizing the acid-lability of said covalent cross-link, said characterizing comprising, (i) subjecting a portion of said enriched HMW sample to partial reduction and alkylation to form a reduced HMW sample; (ii) determining an abundance of said covalent cross-link in acidic conditions, said determining comprising, (I) subjecting a portion of said reduced HMW sample to size exclusion chromatography under denaturing conditions (dSEC) to form an SEC eluate, wherein said dSEC includes an acidic mobile phase; (II) subjecting said SEC eluate to mass spectrometry analysis to determine an abundance of subunit dimers corresponding to said at least one dimer; and (III) using said determination to determine an abundance of said covalent cross-link in acidic conditions; (iii) determining an abundance of said covalent cross-link in non-acidic conditions, said determining comprising, (I) subjecting a portion of said reduced HMW sample to size exclusion chromatography under denaturing conditions (dSEC) to form an SEC eluate, wherein said dSEC includes a non-acidic mobile phase; (II) subjecting said SEC eluate to mass spectrometry analysis to determine an abundance of subunit dimers corresponding to said at least one dimer; and (III) using said determination to determine an abundance of said covalent cross-link in non-acidic conditions; (iv) comparing said abundance of said covalent cross-link in acidic conditions to said abundance of said covalent cross-link in non-acidic conditions to characterize the acid lability of said covalent cross-link; using said first mass measurement, said second mass measurement, and said characterization of the acid-lability of said covalent cross-link to generate a list of at least one potential identity of said covalent cross-link, wherein said at least one potential identity comprises at least two amino acid residues, or variants thereof, capable of forming a covalent cross-link; using said list to identify crosslinked peptides in said enriched HMW sample, said identifying comprising, (i) subjecting a portion of said enriched HMW sample to partial reduction and alkylation to form a reduced HMW sample; (ii) subjecting said reduced HMW sample to dSEC to form at least one fraction including subunit dimers corresponding to said at least one dimer; (iii) collecting said at least one fraction to form a very enriched HMW sample; (iv) contacting said very enriched HMW sample to trypsin to form a peptide digest; (v) contacting said peptide digest to NaBH₃CN to form a reduced peptide digest; (vi) subjecting said reduced peptide digest to reverse phase liquid chromatography-tandem mass spectrometry (RPLC-MS/MS) analysis to obtain peptide masses; and (vii) comparing said peptide masses to predicted masses of crosslinked peptides corresponding to said list of at least one potential identity of said covalent cross-link to identify crosslinked peptides in said enriched HMW sample; and using said identified crosslinked peptides to identify said covalent cross-link.

These, and other, aspects of the invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1A illustrates a post-column denaturation (PCD)-assisted native size exclusion chromatography-mass spectrometry (nSEC-MS) analysis of a protein of interest, according to an exemplary embodiment.

FIG. 1B shows an analysis of covalent and non-covalent HMW species using PCD-assisted nSEC-MS, according to an exemplary embodiment.

FIG. 1C shows a comparison of mass analysis of a covalent dimer and an artificial gas-phase dimer using PCD-assisted nSEC-MS, according to an exemplary embodiment.

FIG. 2A illustrates a workflow for subunit-level analysis of HMW mAb species using PCD-assisted nSEC-MS, according to an exemplary embodiment.

FIG. 2B shows an extracted ion chromatogram (XIC) from subunit-level analysis of mAb-1 HMW sample using PCD-assisted nSEC-MS, according to an exemplary embodiment.

FIG. 2C shows a mass analysis from subunit-level analysis of mAb-1 HMW sample using PCD-assisted nSEC-MS, according to an exemplary embodiment.

FIG. 3 shows a mass analysis of mAb-1 HMW sample using partial reduction treatment and denaturing SEC-MS (dSEC-MS), according to an exemplary embodiment.

FIG. 4A illustrates a workflow of a bottom-up nano LC-MS/MS analysis of crosslinked peptides, according to an exemplary embodiment.

FIG. 4B shows a lysine residue undergoing an oxidation reaction to form a carbonyl group, and a crosslinking reaction with lysine, according to an exemplary embodiment.

FIG. 4C shows a serine residue undergoing an oxidation reaction to form a carbonyl group, and a crosslinking reaction with lysine, according to an exemplary embodiment.

FIG. 4D shows a threonine residue undergoing an oxidation reaction to form a carbonyl group, and a crosslinking reaction with lysine, according to an exemplary embodiment.

FIG. 4E shows an imine bond cross-link undergoing a NaBH₃CN reduction reaction, according to an exemplary embodiment.

FIG. 5A shows a higher-energy collisional dissociation (HCD)-produced MS2 spectrum of a crosslinked peptide, according to an exemplary embodiment.

FIG. 5B shows an electron-transfer dissociation (ETD)-produced MS2 spectrum of a crosslinked peptide, according to an exemplary embodiment.

FIG. 6A shows MS1 spectra of a crosslinked peptide reduced with either NaBH₃CN or NaBD₃CN, according to an exemplary embodiment.

FIG. 6B shows MS1 spectra of a linker-containing product ion reduced with either NaBH₃CN or NaBD₃CN, according to an exemplary embodiment.

### DETAILED DESCRIPTION

Therapeutic monoclonal antibodies (mAbs) often contain high molecular weight (HMW) species that are generated during product manufacturing, shipping, and storage. With sizes ranges from soluble oligomers to visible particles, HMW species could potentially elicit unwanted immunogenic responses, leading to compromised drug safety and efficacy. Therefore, mAb HMW species is an important critical quality attribute (CQA) that needs to be carefully characterized and monitored throughout the product life cycle.

The formation of HMW species is a highly complex process and may occur under different physical (e.g., temperature, agitation, and mechanical contact), chemical (e.g., pH, metal ions, oxidizer, and light exposure), and enzymatic (e.g., lipase-induced degradation of polysorbate) stress conditions. Additionally, an antibody's primary sequence and higher order structure may also affect its susceptibility to interact and aggregate via different mechanisms. The generated HMW species often exhibit different characteristics, including size, interaction nature (covalent or non-covalent), conformation, and modification. Among these characteristics, interaction nature is one of the most desired quality attributes to understand, as it directly affects the reversibility of the HMW species under storage conditions or in vivo. Further, characterizing and understanding the formation mechanism of the interaction, such as covalent cross-links, is of particular interest, because it can offer insights for process improvement to mitigate such HMW species formation.

Conventionally, the assessment of HMW species is often achieved using biophysical techniques and/or liquid chromatography-based methods, such as sedimentation velocity analytical ultracentrifugation (SV-AUC) and size exclusion chromatography (SEC). In particular, SEC with UV detection (SEC-UV) is most widely used for routine product testing due to its simplicity, robustness, and excellent resolving power and quantitative performance in analyzing mAb size variants. Recent developments in direct coupling of SEC to native mass spectrometry (MS) detection has further advanced SEC-based characterization of HMW species. By using MS-compatible buffers that can preserve protein non-covalent interactions, native SEC-MS can provide simultaneous separation and identification of mAb size variants using accurate mass measurement. Further, a post-column denaturation (PCD) assisted nSEC-MS strategy was recently reported that enables improved HMW analysis by allowing differential detection and analysis of both non-covalent and covalent interactions. Specifically, by dissociating the SEC-resolved, non-covalent mAb HMW species into constituent components prior to MS detection, this strategy not only can confirm the constituent subunits of the non-covalent species, but also achieves more accurate mass measurement of the covalent HMW species.

However, despite the many efforts towards characterizing mAb HMW species at the global level, very few have been reported on understanding the interaction or crosslinking chemistry at the local residue level. This is likely due to the often low abundance of HMW species in therapeutic antibodies and their extreme heterogeneity in formation nature, crosslinking types, and sites. Additionally, MS-based approaches for the analysis of crosslinked peptides frequently face the challenge of suboptimal ionization efficiency and inefficient product ion generation using common MS/MS strategies. Furthermore, commercially available software is very inefficient at the identification of crosslinked peptides with unspecified linker mass and crosslinking residues, which further limits the untargeted approach in identifying unknown cross-links.

This disclosure provides advanced MS-based techniques for detection and identification of a cross-link responsible for the formation of HMW species in a protein of interest. In particular, the Examples set forth below describe in detail the identification of a novel cross-link responsible for the formation of covalent HMW species in an IgG molecule. PCD-assisted nSEC-MS analysis was first performed at both intact and subunit levels to measure the mass change associated with this covalent crosslinking in mAb dimers and determine domains of interaction. Following this analysis, denaturing SEC-MS (dSEC-MS) analysis of inter-chain-reduced sample was conducted to allow chromatographic separation of covalent dimers from the sample for MS detection, as well as enable its further enrichment from the bulk HMW material. Combining acid treatment and dSEC-MS analysis of the HMW sample, the exemplary cross-link was also found to be acid-labile, which later became important information for its structural elucidation. Subsequent bottom-up analysis utilized an optimized sample reduction strategy to stabilize the cross-link prior to LC-MS/MS and enabled identification of its chemical structure and the location of the crosslinked amino acid residues. These Examples demonstrate the power of the disclosed MS techniques and their utility in interrogating the chemical nature of covalent HMW species in therapeutics proteins.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein of interest can be a recombinant protein, an in vivo product of gene therapy, a therapeutic protein, an antibody, a bispecific antibody, a multispecific antibody, antibody fragment, monoclonal antibody, antigen-binding protein, fusion protein, scFv, a multisubunit protein, a receptor, a receptor ligand, and combinations thereof.

As used herein, the term “therapeutic protein” refers to any protein that can be administered to a subject for the treatment of a disease or disorder. A therapeutic protein may be any protein with a pharmacological effect, for example, an antibody, a soluble receptor, an antibody-drug conjugate, an antigen-binding protein, or an enzyme. In some exemplary embodiments, the therapeutic protein can be a monoclonal antibody.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. An amino acid consensus sequence may be defined based on a side-by-side analysis of two or more CDRs. The term “antibody,” as used herein, also includes antigen-binding fragments of full antibody molecules.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical crosslinkers, and genetic approaches utilizing recombinant DNA technology.

As used herein, “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, phage display technologies, gene therapy, or a combination thereof.

In some exemplary embodiments, the protein of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS—C—I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, BI cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

In some exemplary embodiments, the sample including the protein of interest can be prepared prior to or following enrichment steps, separation steps, and/or analysis steps. Preparation steps can include alkylation, reduction, denaturation, and/or digestion.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some exemplary embodiments, non-reduced preparation may be used, for example, in order to preserve an endogenous disulfide bond between Fab arms of an antibody or antibody-derived protein. In other exemplary embodiments, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

In some exemplary embodiments, IdeS or a variant thereof is used to cleave an antibody below the hinge region, producing an Fc fragment and a Fab2 fragment. Digestion of an analyte may be advantageous because size reduction may increase the sensitivity and specificity of characterization and detection of the analyte using LC-MS. When used for this purpose, digestion that separates out an Fc fragment and keeps a Fab2 fragment for analysis may be preferred. This is because variable regions of interest, such as the complementarity-determining region (CDR) of an antibody, are contained in the Fab2 fragment, while the Fc fragment may be relatively uniform between antibodies and thus provide less relevant information. Alternatively, or additionally, digestion that separates out a Fab2 fragment and keeps an Fc fragment for analysis may be preferred, because the Fc fragment contains an N-glycosylation site of interest.

IdeS digestion has a high efficiency, allowing for high recovery of an analyte. The digestion and elution process may be performed under native conditions, allowing for simple coupling to a native LC-MS system. IdeS or variants thereof are commercially available and may be marketed as, for example, FabRICATOR® or FabRICATOR Z®.

As used herein, the terms “oxidative species,” “OS,” or “oxidation variant” refer to the variants of a protein formed by oxidation. Oxidation variants can result from oxidation occurring at, for example, proline, arginine, lysine, glutamic acid, aspartic acid, threonine, serine, histidine, cysteine, methionine, tryptophan, phenylalanine and/or tyrosine residues.

As used herein, a “sample” can be obtained from any step of the bioprocess, such as cell culture fluid (CCF), harvested cell culture fluid (HCCF), any step in the downstream processing, drug substance (DS), or a drug product (DP) comprising the final formulated product. In some other specific exemplary embodiments, the sample can be selected from any step of the downstream process of clarification, chromatographic production, viral inactivation, or filtration. In some specific exemplary embodiments, the drug product can be selected from manufactured drug product in the clinic, shipping, storage, or handling.

As used herein, the term “impurity” can include any undesirable protein present in a protein sample or protein biopharmaceutical product. Impurity can include process and product-related impurities. The impurity can further be of known structure, partially characterized, or unidentified. Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

Product-related impurities (e.g., precursors, certain degradation products) can be molecular variants arising during manufacture and/or storage that do not have properties comparable to those of the desired product with respect to activity, efficacy, and safety. Such variants may need considerable effort in isolation and characterization in order to identify the type of modification(s). Product-related impurities can include truncated forms, modified forms, and aggregates. Truncated forms are formed by hydrolytic enzymes or chemicals which catalyze the cleavage of peptide bonds. Modified forms include, but are not limited to, deamidated, isomerized, mismatched S—S linked, oxidized, or altered conjugated forms (e.g., glycosylation, phosphorylation). Modified forms can also include any post-translational modification form. Aggregates include dimers and higher multiples of the desired product. (Q6B Specifications: Test Procedures and Acceptance Criteria for Biotechnological/Biological Products, ICH August 1999, U.S. Dept. of Health and Humans Services).

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase (RP) liquid chromatography, ion-exchange (IEX) chromatography, size exclusion chromatography (SEC), affinity chromatography, hydrophobic interaction chromatography (HIC), hydrophilic interaction chromatography (HILIC), or mixed-mode chromatography (MMC).

In some exemplary embodiments, the methods of the present invention include the use of size exclusion chromatography. Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase. Variants of a protein of interest that have a higher molecular weight or lower molecular weight than the main species, or than the intended product, may be referred to as “size variants.”

Analytes eluting from an SEC column may be separated into fractions based on elution time. For example, analytes eluting earlier than the functional form of a protein of interest, for example the monomeric form, may be broadly categorized as high molecular weight (HMW) species. A HMW fraction may be further subdivided into, for example, a very high molecular weight (vHMW) fraction and a dimer fraction (representing the elution time of a dimer of the protein of interest). Analytes eluting later than the functional form of a protein of interest may be broadly categorized as low molecular weight (LMW) species, and may be further subdivided into a LMW fraction and a later tail fraction.

A HMW species, such as a dimer, may be formed as a product of a crosslinking reaction. A cross-link is a bond or a short sequence of bonds that links one polypeptide chain to another. Crosslinking may occur naturally or synthetically. Examples of cross-links include, for example, disulfide bonds, amine bonds, ester bonds, or imine bonds. A crosslinking reaction may increase or decrease the total molecular weight of crosslinked molecules relative to the unmodified molecules, depending on the crosslinking mechanism. In an exemplary embodiment, a size variant of a protein of interest is a dimer formed by a cross-link. In a particular exemplary embodiment, the cross-link is an imine bond. In a more particular exemplary embodiment, the imine bond is formed between a lysine residue and a serine residue.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Porous chromatographic resins appropriate for size-exclusion chromatography of viruses may be made of dextrose, agarose, polyacrylamide, or silica which have different physical characteristics. Polymer combinations can also be also used. Most commonly used are those under the tradename “SEPHADEX” available from Amersham Biosciences. Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, Calif.).

In some exemplary embodiments, the mobile phase used to obtain said eluate from size exclusion chromatography can comprise a volatile salt. In some specific embodiments, the mobile phase can comprise ammonium acetate, ammonium bicarbonate, or ammonium formate, or combinations thereof.

Online coupling of SEC with direct MS detection under near native conditions (native SEC-MS) has gained a lot of interest over the past few years to study mAb HMW species (Rouby et al., supra; Ehkirch A, Hernandez-Alba O, Colas O, Beck A, Guillarme D, Cianferani S. Hyphenation of size exclusion chromatography to native ion mobility mass spectrometry for the analytical characterization of therapeutic antibodies and related products. J Chromatogr B Analyt Technol Biomed Life Sci 2018:1086 (176-183); Haberger M, Leiss M, Heidenreich A K, Pester O, Hafenmair G, Hook M, Bonnington L, Wegele H, Haindl M, Reusch D et al. Rapid characterization of biotherapeutic proteins by size-exclusion chromatography coupled to native mass spectrometry. MAbs 2016:8(2): 331-339. Using MS-compatible mobile phases that can preserve protein conformation and non-covalent interactions, native SEC-MS (nSEC-MS) can provide rapid and improved identification of size variants based on accurate mass measurement.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device. The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “mass analyzer” includes a device that can separate species, that is, atoms, molecules, or clusters, according to their mass. Non-limiting examples of mass analyzers that could be employed are time-of-flight (TOF), magnetic electric sector, quadrupole mass filter (Q), quadrupole ion trap (QIT), orbitrap, Fourier transform ion cyclotron resonance (FTICR), and also the technique of accelerator mass spectrometry (AMS).

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, the mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

In some exemplary embodiments, a mass spectrometer may use one or more of various fragmentation or analysis techniques, including, for example, collision-induced dissociation (CID), electron-transfer dissociation (ETD), electron-transfer/collision-induced dissociation (ETciD), electron-transfer/higher-energy collisional dissociation (EThcD), or ultra-violet photodissociation (UVPD).

In some exemplary embodiments, LC-MS, such as SEC-MS, can be performed under native conditions. As used herein, the term “native conditions” can include performing mass spectrometry under conditions that preserve non-covalent interactions in an analyte. Native mass spectrometry is an approach to study intact biomolecular structure in the native or near-native state. The term “native” refers to the biological status of the analyte in solution prior to subjecting to the ionization. Several parameters, such as pH and ionic strength, of the solution containing the biological analytes can be controlled to maintain the native folded state of the biological analytes in solution. Commonly, native mass spectrometry is based on electrospray ionization, wherein the biological analytes are sprayed from a nondenaturing solvent. Other terms, such as noncovalent, native spray, electrospray ionization, nondenaturing, macromolecular, or supramolecular mass spectrometry can also be describing native mass spectrometry. In exemplary embodiments, native MS allows for better spatial resolution compared to non-native MS, improving detection of biotransformation products of a therapeutic protein. For detailed review on native MS, refer to the review: Elisabetta Bocri Erba & Carlo Pe-tosa, The emerging role of native mass spectrometry in characterizing the structure and dynamics of macromolecular complexes, 24 PROTEIN SCIENCE 1176-1192 (2015).

In some exemplary embodiments, the methods of the present invention may include post-column denaturation (PCD). Denaturing analytes subsequent to chromatographic separation but prior to mass analysis provides the benefits of native chromatographic separation as described above, and additionally improves protein or peptide characterization by reducing interference from co-eluting, non-covalent species. PCD may be achieved by introducing a post-column denaturant flow into a chromatographic column flow using a T-mixer. The denaturing solvent must be selected based on compatibility with direct MS detection, and the ability to disrupt the majority of non-covalent interactions immediately after post-column mixing. Suitable post-column denaturants include, for example, acetonitrile and formic acid. Exemplary methods for post-column denaturation are described in Yan et al., Post-Column Denaturation-Assisted Native Size-Exclusion Chromatography-Mass Spectrometry for Rapid and In-Depth Characterization of High Molecular Weight Variants in Therapeutic Monoclonal Antibodies, J. AM SOC MASS SPECTROM 32, 2885-2894 (2021), which is incorporated by reference in its entirety.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenrescarch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

It is understood that the present invention is not limited to any of the aforesaid protein(s), antibody(s), monoclonal antibody(s), bispecific antibody(s), protein expression system(s), oxidation species, cross-link(s), high molecular weight species, protein alkylating agent(s), protein denaturing agent(s), protein reducing agent(s), digestive enzyme(s), hydrolyzing agent(s), sample(s), liquid chromatography system(s), mobile phase(s), mass spectrometer(s), database(s), or bioinformatics tool(s), and any protein(s), antibody(s), monoclonal antibody(s), bispecific antibody(s), protein expression system(s), oxidation species, cross-link(s), high molecular weight species, protein alkylating agent(s), protein denaturing agent(s), protein reducing agent(s), digestive enzyme(s), hydrolyzing agent(s), sample(s), liquid chromatography system(s), mobile phase(s), mass spectrometer(s), database(s), or bioinformatics tool(s) can be selected by any suitable means.

The present invention will be more fully understood by reference to the following Examples. They should not, however, be construed as limiting the scope of the invention.

### EXAMPLES

Materials. Deionized water was provided by a Milli-Q integral water purification system installed with a MilliPak Express 20 filter (MilliporeSigma, Burlington, MA). Formic acid, trifluoroacetic acid, dithiothreitol, iodoacetamide, and acetonitrile (LC-MS grade) were purchased from Thermo Fisher Scientific (Waltham, MA). Ammonium acetate (LC-MS grade), acetic acid (LC-MS grade), urea, iodoacetamide (IAA), NaBH 3CN, and NaBD₃CN were purchased from Sigma Aldrich (St. Louis, MO). Trypsin/rLysC was purchased from Promega (Madison, WI). FabRICATOR (IdeS) protease was purchased from Genovis (Cambridge, MA).

Sample preparation. mAb-1 was produced in CHO cells at Regeneron Pharmaceuticals. The enriched HMW sample of mAb-1 was generated by fractionating the HMW species from the mAb-1 drug substance (DS) sample using a semi-preparation scale SEC column. The final enriched HMW sample contains roughly 67% dimer and 33% monomer. Prior to PCD-assisted nSEC-MS, the mAb-1 HMW sample was treated with PNGase F (1 IUB milliunit per 10 μg of protein) at 45° C. in 50 mM Tris-HCl (pH 7.0) for 1 hour to remove the N-glycan chains from each heavy chain CH2 domain for intact level analysis. For subdomain analysis, an aliquot of the deglycosylated mAb-1 HMW sample was digested with FabRICATOR (1 IUB milliunit per 1 μg of protein) in 50 mM Tris-HCl (pH 7.5) at 37° C. for 1 hour to generate F(ab)′₂ and Fc fragments. Limited reduction was then performed on the digested sample by incubating with 2 mM DTT in 50 mM Tris-HCl (pH 7.5) at 37° C. for 30 min to reduce inter-chain disulfide bonds. For dSEC-MS analysis, an aliquot of deglycosylated mAb-1 HMW sample was subjected to the same limited reduction treatment described above to remove the inter-chain disulfide bonds and generate HC and LC. To further enrich the covalent crosslinked HMW species for bottom-up analysis, the “HMW” peak from dSEC-UV analysis was fractionated from 100 μg of starting enriched HMW material and neutralized (to pH 8.0) using 1 M ammonia solution immediately upon fraction collection.

dSEC-MS. Denaturing size exclusion chromatography (dSEC) was performed on a Waters Acquity I-Class UPLC system (Waters, Milford, MA) equipped with an Acquity BEH200 SEC column (4.6×150 mm, 200 Å, 1.7 μm, Waters, Milford, MA) with the column compartment set to 30° ° C. An isocratic flow of mobile phase containing 70% water, 30% acetonitrile, 7.5 mM ammonium acetate, and 0.075% formic acid operated at 0.2 mL/min was applied to separate and elute protein size variants. 5-20 μg of mAb sample was used for each analysis, and separated size variants were monitored by UV (280 nm) using a photodiode array (PDA) detector (Waters, Milford, MA) or by MS using a Q-Exactive Plus mass spectrometer equipped with a heated electrospray ion source (HESI, Thermo Fisher Scientific, Waltham, MA). For MS detection, the instrument source parameters were set as following: spray voltage 3.5 kV, sheath gas 20 (arbitrary units), Aux gas 10, capillary temperature 350° ° C., Aux gas temperature 250° C., and S-lens RF level 60. Full MS scan was performed with instrument resolving power set to 17500 and scan range between 2000-5500.

PCD-assisted nSEC-MS. Native SEC chromatography was performed on an UltiMate 3000 UHPLC System (Thermo Fisher Scientific, Bremen, Germany) equipped with an Acquity BEH200 SEC column (4.6×300 mm, 1.7 μm, 200 Å; Waters, Milford, MA) with the column compartment set to 30° ° C. An isocratic flow of 150 mM ammonium acetate at 0.2 mL/min was applied to separate and elute protein size variants. To enable post-column denaturation, a denaturing solution consisting of 60% ACN, 36% water, and 4% FA was delivered by a secondary pump at a flow rate of 0.2 mL/min and then mixed with the SEC eluent (1:1 mixing) using a T-mixer before being subjected to MS detection. To enable online native MS analysis, the combined analytical flow (0.4 mL/min) was split into a microflow (<10 μL/min) for nano-electrospray ionization (NSI)-MS detection and a remaining high flow for UV detection. A Thermo Q Exactive UHMR (Thermo Fisher Scientific, Bremen, Germany) equipped with a Microflow-Nanospray Electrospray Ionization (MnESI) Source and a Microfabricated Monolithic Multi-nozzle (M3) emitter (Newomics, Berkley, CA) was used for native MS analysis. A detailed experimental setup and instrument parameters are described in Yan Y, Xing T, Wang S, Li N. Versatile, sensitive, and robust native LC-MS platform for intact mass analysis of protein drugs. J Am Soc Mass Spectrom 2020:31(10): 2171-2179, which is incorporated by reference in its entirety. To disable PCD, the flow of the denaturing solution was set to zero.

Trypsin digestion and bottom-up analysis. The dSEC-fractionated HMW sample was dried in a CentriVap centrifugal vacuum concentrator (Labconco, Kansas City, MO), followed by reconstitution using a buffer containing 8 M urea, 5 mM DTT, and 100 mM HEPES (pH 7.5) and incubation at 50° ° C. for 30 minutes. The denatured and reduced sample was then alkylated with 12.5 mM IAA at room temperature in the dark for 30 minutes. After alkylation, the sample was 8-fold diluted with 100 mM HEPES (pH 7.5) and mixed with trypsin/rLysC at a 1:20 enzyme:protein ratio. The sample was digested at 37° C. for 4 hours. Digestion was halted by adding formic acid to 0.2% before reverse phase (RP) LC-MS/MS analysis. For samples treated with NaBH₃CN or NaBD₃CN, 100 mM NaBH 3CN or NaBD₃CN was added to the sample immediately after trypsin/rLysC digestion and incubated at room temperature for 1 hour prior to the addition of formic acid.

Reverse-phase nano LC-MS/MS analysis of the protein digests was performed on an UltiMate 3000 RSLCnano LC connected to an Orbitrap Fusion Lumos Tribrid mass spectrometer. Peptides were separated on a C18 column (1.7 μm, 75 μm×25 cm) (CoAnn Technology, Richland, WA) by a 100 minute gradient with 0.1% FA in water as mobile phase A, and 80% acetonitrile, 19.92% water, 0.08% formic acid as mobile phase B (0-10 min, 4% B; 10-80 min, 4-38% B; 80-81 min, 38-95% B; 81-100 min, 95% B). The mobile phase flow rate was 0.25 μL/min. The nano ESI spray voltage was set to 2.1 kV. MS analysis was performed in data-dependent acquisition (DDA) mode with a cycle time of 2 seconds. For MS1 scan, the following instrument parameters were used: Orbitrap resolution 120000, scan range 350-2000, maximum injection time 50 ms, and standard AGC target. For data dependent MS2 analysis, the following scan parameters were used: higher-energy collisional dissociation (HCD) energy 30%, Orbitrap resolution 30000, maximum injection time 50 ms, standard AGC target, dynamic exclusion 30 s, and analyzed charge state 2-7. For targeted MS/MS fragmentation by electron transfer dissociation (ETD), analysis was performed on the targeted peptide of +4 and +5 charge states using calibrated, charge-dependent ETD parameters.

Data analysis. Intact mass spectra from PCD-assisted nSEC-MS analysis and dSEC-MS analysis were deconvoluted using Intact Mass™ software from Protein Metrics. Database search of peptide mapping data was performed using Byonic™ software and manually examined using Byologic™ software from Protein Metrics.

### Example 1. Detection of an Unknown Covalent Cross-Link in mAb Species by PCD-Assisted nSEC-MS Analysis

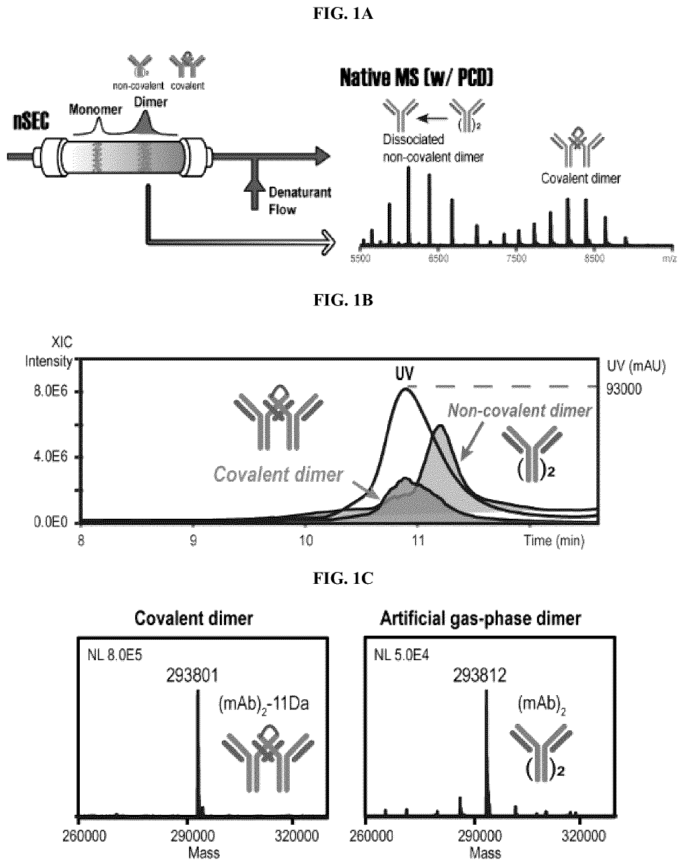

In-depth characterization of HMW species is highly desirable during the development of mAb drug products, as it can provide deep understanding of the aggregation mechanism and frameworks for risk assessment of the HMW species. A recently described PCD-assisted SEC-MS method is a powerful tool to perform both intact and subunit level characterization of mAb HMW species. Specifically, this method employed a denaturing solvent after SEC separation to dissociate SEC-resolved, non-covalent mAb HMW species into constituent components prior to MS detection. As a result, mass measurement of the SEC-resolved HMW peak consists of 1) the covalent HMW species and 2) the constituent subunits dissociated from the non-covalent HMW complexes, as shown in FIG. 1A. In particular, because of the removal of interference from co-eluting, non-covalent species, more accurate mass measurement of non-dissociable HMW species can be achieved using this approach. As demonstrated in FIG. 1B, using nSEC-MS analysis, the predominant HMW peak in the enriched HMW sample of mAb-1 was readily identified as mAb-1 dimer species. Application of PCD then revealed the presence of both the non-covalent dimer (FIG. 1B, orange trace, represented by the XIC of mAb-1 monomer signal under PCD conditions) and the covalent dimer (FIG. 1B, blue trace, represented by the XIC of non-dissociated mAb-1 dimer signal under PCD conditions). Accurate mass measurement revealed that the covalent mAb-1 dimer exhibited a mass decrease of approximately 11 Da compared to an artificial non-covalent mAb-1 dimer (formed in the gas-phase under electrospray source condition), as shown in FIG. 1C, suggesting the potential presence of a covalent cross-link with a negative delta mass. However, considering the relatively low resolving power of the orbitrap mass analyzer at higher m/z regions for the large mAb dimer species (m/z 7000-9000, as shown in FIG. 1A), the accuracy of the measured mass of this covalent cross-link may be non-ideal.

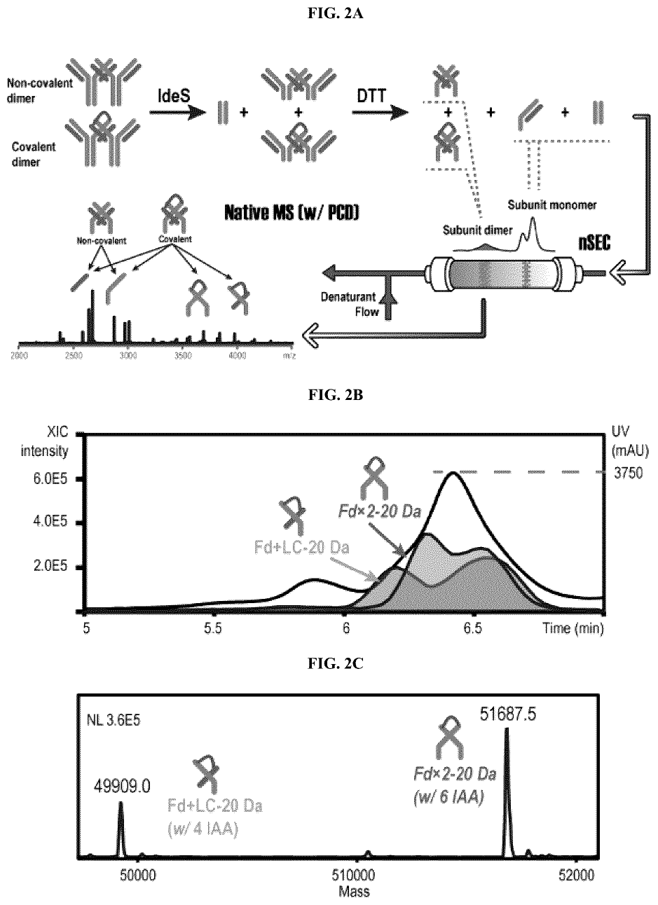

To achieve better mass accuracy for measurement of the covalent dimers, subunit level analysis for the mAb-1 dimer was also performed. First, the enriched mAb-1 HMW sample was digested with IdeS, which cleaves the mAb molecule under the hinge region, to release F(ab)′₂ and Fc (consisting of two non-covalently interacting Fc/2) fragments from both monomeric and dimeric mAb-1 species in the sample, as shown in FIG. 2A. Following this, partial reduction and alkylation was applied to disrupt the inter-chain disulfide bonds (HC-HC and HC-LC) and further reduce F(ab)′₂ to Fab (consisting of non-covalently interacting Fd and LC subunits). As a result, the mAb-1 enriched HMW sample was converted into a mixture containing Fab and Fc monomers and dimers (including possibly Fab dimer, Fc dimer, and Fab-Fc dimer). Similar to the intact level analysis described above, subsequent PCD-assisted nSEC-MS analysis could then provide accurate mass measurement of the covalent dimer species by 1) chromatographically resolving the sub-domain dimers from monomers and 2) removing non-covalent interferences for MS detection. Specifically, due to the absence of inter-chain disulfide bonds between Fd and LC, the non-covalent dimers were dissociated into subunit fragments (i.e., Fd, LC, Fc/2), while covalent dimers may produce crosslinked subunits between Fd, LC and Fc/2, as shown in FIG. 2A. In this case, analysis of the digested mAb-1 HMW sample resulted in the detection of two covalently crosslinked dimers, including Fd homodimer and Fd-LC heterodimer, in the SEC-resolved dimer region, as shown in FIG. 2B. Both detected masses suggested a covalent cross-link of approximately −20 Da, as shown in FIG. 2C, which largely agreed with the measured value at the intact level (−11 Da). Notably, as the mass resolving power of orbitrap mass analyzer is inversely proportional to the m/z, e.g., size of the analyte, mass measurement of the crosslinked dimer at the subunit level is expected to be more accurate than that at the intact level. Therefore, during the further investigation of this unknown cross-link, a delta mass of approximately −20 Da was considered.

### Example 2. Characterization of the Acid-Labile Nature of a Covalent mAb Cross-Link

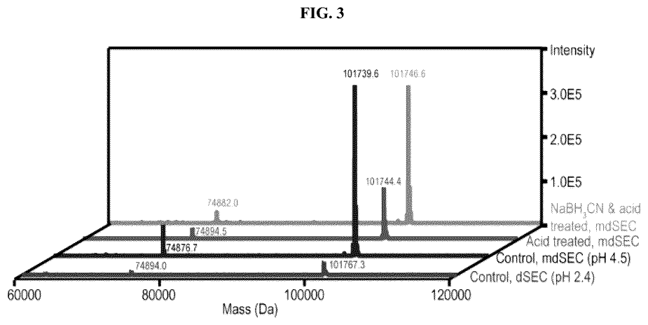

Denaturing SEC-MS is another useful tool for studying mAb covalent dimers, as it can effectively dissociate non-covalent interactions and thus chromatographically separate covalent dimers from monomers and non-covalent dimers. In particular, when combined with partial reduction treatment (inter-chain disulfide disruption) to reduce the analyte size to HC, LC, and corresponding dimers, dSEC-MS can provide valuable information on the crosslinking at the individual chain level. In this case, the partially reduced mAb-1 HMW sample was first subjected to a conventional dSEC condition with mobile phase composed of 30% ACN and pH adjusted to 2.4, followed by online MS analysis. Surprisingly, very low levels of HC homodimers or HC-LC heterodimers were detected using this approach, as shown in FIG. 3 (green trace), which was contradictory to what was observed by PCD-assisted nSEC-MS analyses, as described earlier. To explain the observed discrepancy, it was proposed that the −20 Da-associated cross-link may be susceptible to degradation under strong acidic conditions.

To test this hypothesis, a modified denaturing mobile phase buffered at pH 4.5 (0.075% FA, 7.5 mM ammonium acetate, 70% water, 29.9% ACN) was used to perform dSEC-MS analysis for partially reduced mAb-1 HMW sample. As a result, significantly higher levels of HC homodimer and HC-LC heterodimer were detected, as shown in FIG. 3 (black trace), both of which displayed approximately 20 Da mass decrease from the corresponding non-covalent dimer forms.

To further confirm this result, the partially reduced mAb-1 HMW sample was also treated with 0.1% FA (pH 2.7, 30 minutes) prior to dSEC-MS analysis using the modified mobile phase. This treatment was also applied to all dSEC conditions described below, unless otherwise noted. The detected HMW species exhibited an approximately 70% decrease in abundance compared to an untreated sample, further supporting the finding of the acid-labile nature of the −20 Da-associated cross-link, as shown in FIG. 3 (magenta trace).

Among the several different cross-links reported in protein crosslinking studies, including amide, ester, imine, and radical induced covalent bond, only imine is susceptible to hydrolysis at low pH, which has not previously been reported in mAb molecules. Considering the unique characteristics of imine being readily reducible by NaBH₃CN, the partially reduced mAb-1 HMW sample was also incubated with NaBH 3CN before being subjected to acid treatment (0.1% FA). The resulting HMW species analyzed by dSEC-MS indicated that most of the crosslinked HMW species became resistant to acid-induced hydrolysis after NaBH₃CN treatment and were preserved for MS detection, as shown in FIG. 3 (blue trace). Together, these findings suggested that the −20 Da-associated cross-link was likely to be an imine bond.

### Example 3. Identification of a Covalent mAb Cross-Link Using NaBH₃CN Reduction and Bottom-Up Analysis

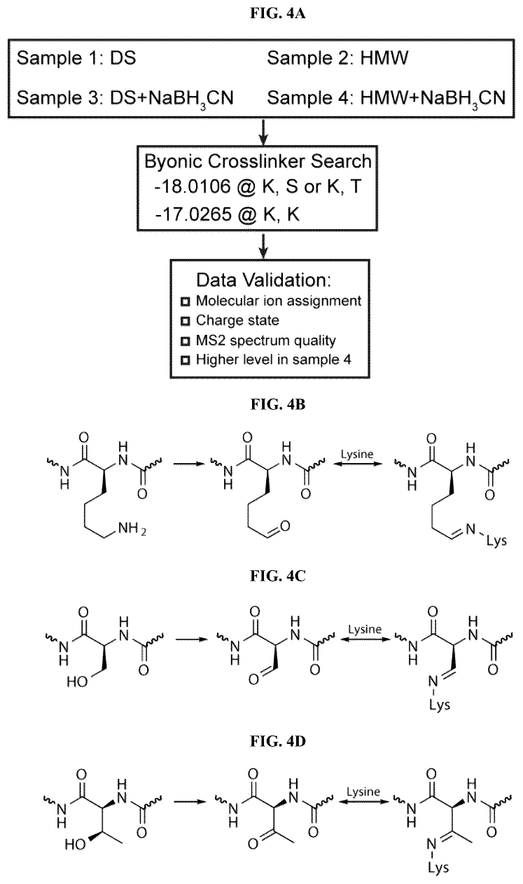

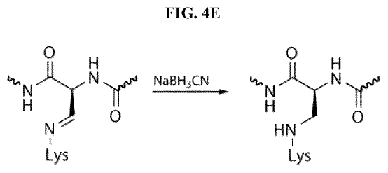

To confirm the proposed identity of the unknown cross-link and determine the hot spots along the protein sequence for this crosslinking reaction, bottom-up analysis was applied to study the crosslinked peptides in mAb-1 HMW sample. Considering the likely high heterogeneity of crosslinking sites and low abundance of individual crosslinked peptides, the enriched HMW sample was first further enriched by partial reduction and dSEC fractionation of the crosslinked HC-HC and HC-LC dimers. The generated HMW material, together with mAb-1 unenriched DS sample (containing >99% mAb-1 monomer), were subjected to trypsin digestion and NaBH₃CN treatment (for sample 3 and 4 only), followed by nano LC-MS/MS analysis, as shown in FIG. 4A. Among these four samples analyzed, the DS samples (sample 1 and 3) were included as negative controls to show that crosslinked peptides were not present in DS and cannot be introduced by the NaBH 3CN treatment. The HMW sample without NaBH₃CN treatment (sample 2) was also not expected to produce meaningful levels of crosslinked peptides, due to imine bond degradation under low pH conditions applied at digestion end and during reverse phase analysis (containing 0.1% FA). In contrast, HMW sample with NaBH₃CN treatment (sample 4) to reduce and stabilize the imine bonds was expected to produce the highest level of crosslinked peptides, which can be used for cross-link identification and characterization.

In protein crosslinking, the imine bond is usually formed from a condensation reaction between a primary amine group and a carbonyl group, which is associated with an 18 Da mass loss. While the primary amine is readily available from the side chain of lysine residues (or the protein N-terminus), the carbonyl group does not exist in natural amino acids and can only be generated from another chemical reaction on the existing functional groups. For example, oxidation of amino acid residues, including proline, arginine, lysine, glutamic acid, aspartic acid, threonine, and serine, by metal catalyzed reactive oxygen species can all lead to the formation of carbonyl groups. In this case, since the cross-link was measured at approximately −20 Da, of which −18 Da is attributable to the reaction between primary amine and carbonyl groups, the carbonyl generation reaction was expected to only cause minimal mass change (approximately −2 Da) to the amino acids. This narrowed down the list of amino acids for generating the carbonyl group to lysine (side chain oxidation forming allysine, −1 Da, as shown in FIG. 4B), serine, and threonine (converting side chain hydroxyl group to carbonyl group, −2 Da, as shown in FIG. 4C and FIG. 4D). The most commonly, if not the only, reported imine bond as a protein cross-link was formed between the side chains of lysine (free amine) and allysine (carbonyl).

Considering their close crosslinking-associated mass changes compared to that observed for the unknown cross-link, all three potential crosslinking mechanisms (FIG. 4B, FIG. 4C, and FIG. 4D) were searched for during bottom-up analysis of the four samples, as shown in FIG. 4A. It should be noted that, as the NaBH 3CN reduction reaction of an imine bond increases the mass by 2 Da, as shown in FIG. 4E, the corresponding masses of the cross-links used in the search were also 2 Da higher than those of the imine bond cross-links.

The resulting list of identified crosslinked peptides were then manually inspected by applying more stringent criteria in MS1 isotope pattern, MS2 spectral quality, and chromatographic peak shape, to remove low-confidence identifications. The abundances of the remaining crosslinked peptides were also evaluated and compared among the four samples to select only those exhibiting the highest abundance in Sample 4 as true crosslinking identifications. In total, five crosslinked peptides with serine to lysine (S-K) imine cross-links were successfully identified with high confidence, as shown in Table 1 (with the relevant serines and lysines indicated as “S*” and “K*”).

TABLE 1
Identification of crosslinked peptides

| Crosslinked | | |
|---|---|---|
| Peptide | Peptide 1 | Peptide2 |
| 1 | EVQLVES*GGGLEQPGGSLR | GLEWVSSIXXXXXXTYYADSVK*G |
| | | R |
| 2 | EVQLVES*GGGLEQPGGSLR | DNSK*NTLYLQMNSLR |
| 3 | GLEWVS*SIXXXXXXTYYADSV | DNSK*NTLYLQMNSLR |
| | K | |
| 4 | GLEWVS*SIXXXXXXTYYADSV | VSNK*GLPSSIEK |
| | K | |
| 5 | STS*ESTAALGCLVK | VDK*R |

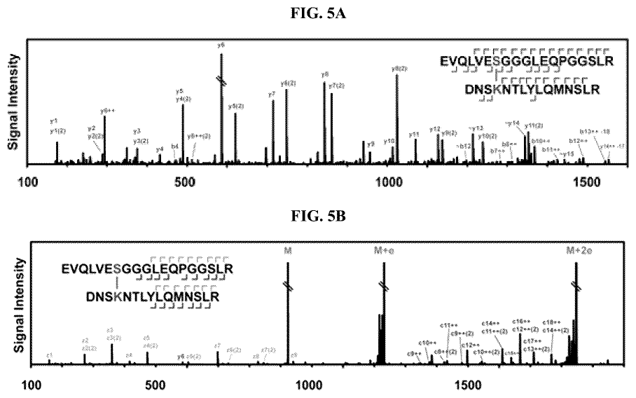

No crosslinked peptides that associated with either lysine to allysine (K-K) or threonine to lysine (T-K) crosslinking reactions were identified. A representative HCD-produced MS2 spectrum of a selected crosslinked peptide (peptide 2 from Table 1) is shown in FIG. 5A, depicting the assignments of both peptide sequences and crosslinking sites by employing product ion detection. An alternative fragmentation strategy using ETD was also employed to generate complementary product ions to those generated by HCD, and showed consistent assignments, as shown in FIG. 5B. In particular, ETD fragmentation of the parent crosslinked peptide generated more product ions containing the linker portion of the dipeptide, thus further supporting the assignment of the S-K crosslinking.

Despite extensive enrichment of the mAb-1 covalent HMW species prior to bottom-up analysis, only five crosslinked peptides were identified with high confidence. This is likely attributable to 1) the high heterogeneity in crosslinking sites between the 87 serine residues and 39 lysine residues in mAb-1 HC and LC; and 2) insufficient MS sensitivity and fragmentation efficiency for detecting crosslinked peptides. To provide further insights on the heterogeneity of crosslinked sites in mAb-1, a database search was conducted on the bottom-up results for oxidized serine species, which is the intermediate product before imine bond formation. As listed in Table 2, a total of 20 oxidized serine residues (indicated below as “s”) were detected with high confidence at meaningful levels along the sequence of mAb-1.

TABLE 2
Identification of oxidized serine residues

| | | Heavy | | | | |
|---|---|---|---|---|---|---|
| | | Chain/ | Start | End | | |
| Peptide | | Light | Amino | Amino | Serine | |
| Number | Sequence | Chain | Acid | Acid | Position | Abundance |
| 1 | R.DNsKNTLYLQMNS | Heavy | 73 | 87 | 75 | 0.01% |
| | LR.A | Chain | | | | |
| 2 | - | Heavy | 1 | 19 | 7 | 0.14% |
| | .EVQLVEsGGGLEQPG | Chain | | | | |
| | GSLR.L | | | | | |
| 3 | K.GFYPSDIAVEWEsN | Heavy | 376 | 397 | 388 | 0.15% |
| | GQPENNYK.T | Chain | | | | |
| 4 | K.GLEWVSSISGsGGN | Heavy | 44 | 65 | 54 | 0.13% |
| | TYYADSVK.G | Chain | | | | |
| 6 | K.GLEWVsSISGSGGN | Heavy | 44 | 65 | 49 | |
| | TYYADSVK.G | Chain | | | | |
| 7 | K.GLEWVSsISGSGGN | Heavy | 44 | 65 | 50 | |
| | TYYADSVK.G | Chain | | | | |
| 8 | K.GLEWVSSIsGSGGN | Heavy | 44 | 65 | 52 | |
| | TYYADSVK.G | Chain | | | | |
| 10 | K.GPSVFPLAPCsR.S | Heavy | 130 | 141 | 140 | 0.04% |
| | | Chain | | | | |
| 14 | R.STsESTAALGCLVK. | Heavy | 142 | 155 | 144 | 0.18% |
| | D | Chain | | | | |
| 15 | K.TTPPVLDsDGSFFL | Heavy | 398 | 414 | 405 | 0.01% |
| | YSR.L | Chain | | | | |
| 18 | R.WQEGNVFSCsVMH | Heavy | 422 | 444 | 431 | 0.99% |
| | EALHNHYTQK.S | Chain | | | | |
| 20 | R.WQEGNVFsCSVMH | Heavy | 422 | 444 | 429 | |
| | EALHNHYTQK.S | Chain | | | | |
| 11 | K.SGTAsVVCLLNNFY | Light | 132 | 147 | 136 | 0.17% |
| | PR.E | Chain | | | | |
| 12 | R.SSQSLLYsIGYNYL | Light | 25 | 44 | 32 | 0.10% |
| | DWYLQK.S | Chain | | | | |
| 16 | K.VDNALQsGNSQES | Light | 155 | 174 | 161 | 0.02% |
| | VTEQDSK.D | Chain | | | | |
| 17 | K.VYACEVTHQGLsSP | Light | 196 | 212 | 207 | 0.04% |
| | VTK.S | Chain | | | | |

Considering lysine residues are usually located at solvent-accessible regions of the protein, these oxidized serine residues can presumably react with each of the 39 lysine residues, leading to 780 possible crosslinked peptides. These 780 differently crosslinked peptides all share the same Ser-Lys imine cross-link, which collectively contribute to the intact level-detected homogeneous HMW species exhibiting a 20 Da mass decrease. However, when analyzed individually at the peptide level, each pair of crosslinked peptides could be present at extremely low levels, and only those with higher abundances could be detected by the bottom-up MS analysis.

### Example 4. Confirmation of the Identity of an Imine Cross-Link by Isotope Labeling Reduction

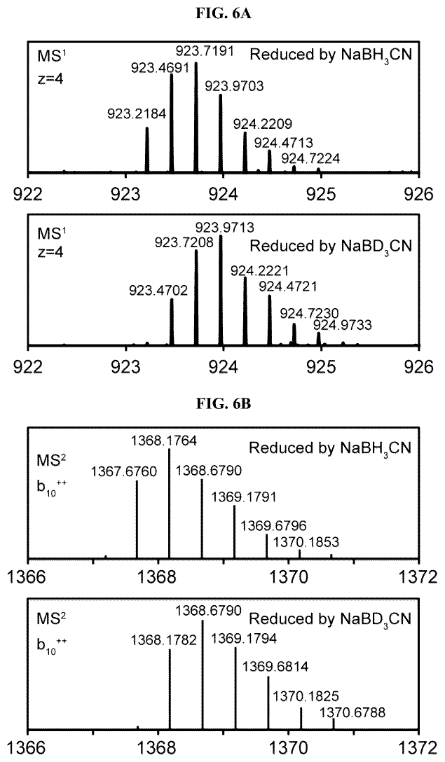

As reduction of the imine bond by NaBH₃CN leads to the addition of two hydrogens that are sourced from NaBH₃CN and solvent water, respectively, NaBH₃CN reduction can be used for further confirmation of the cross-link. For this purpose, two mAb-1 HMW samples were digested and treated with either NaBH 3CN or NaBD₃CN, followed by LC-MS/MS analysis. The resulting MS1 spectra of the same crosslinked peptide (peptide 2 from Table 1) from these two parallel analyses exhibited a 1 Da mass increase from the NaBH₃CN treated sample to the NaBD₃CN treated sample, as shown in FIG. 6A. This 1 Da mass difference of the reaction products is consistent with the expected mass difference between hydrogen and deuterium, hence directly confirming that the crosslinked peptides underwent imine bond reduction by NaBH₃CN treatment. Moreover, the same delta mass of 1 Da was also observed for a linker-containing product ion, b10, of the dipeptide from MS2 analysis, further confirming the occurrence of the reduction reaction at the cross-link, as shown in FIG. 6B. Together, these results demonstrate that S-K crosslinking via serine oxidation and subsequent imine bond formation with the side chain of lysine was the main cause of covalent HMW species in mAb-1 sample.

Characterization of HMW species and understanding their forming mechanisms are highly important during the development of therapeutic mAb products. The development of a PCD-assisted SEC-MS strategy was recently reported that enables improved HMW size variant analysis by dissociating SEC-resolved non-covalent species into constituent components before MS detection. As a result, more accurate mass measurements of the undissociated covalent HMW species can be achieved and used to support further study in understanding the covalent cross-link chemistry.

In these examples, a PCD-assisted nSEC-MS approach was first applied to intact level analysis of mAb-1 enriched HMW sample, and successfully detected a novel covalent cross-link that formed mAb-1 dimers. Subsequent subunit level analysis using IdeS digestion and partial reduction (inter-chain disulfide bond disruption) confirmed this covalent cross-link and determined its associated mass change of approximately −20 Da. Further, dSEC-MS analysis of the inter-chain-reduced HMW sample, operated using mobile phases with varying pH, revealed the acid-labile nature of this novel cross-link. NaBH₃CN treatments then showed that this cross-link could be stabilized against acid degradation by NaBH₃CN reduction, suggesting its likely imine nature. Considering the measured mass change of approximately −20 Da for the covalent dimers, three possible imine bond-based covalent cross-links were proposed and applied in database search during the subsequent bottom-up analysis of the mAb-1 HMW sample. Through further enrichment of the covalent HMW sample prior to trypsin digestion and inclusion of three negative control samples, bottom-up analysis with targeted data base search successfully identified several crosslinked peptides with Ser-Lys imine cross-link. Additionally, the identity of imine cross-link was further confirmed by performing NaBH₃CN reduction using heavy and light isotope labeled reagents.

It is important to note that, although this disclosure provides the example of a mAb-1 sample comprising this novel Ser-Lys imine cross-link, it is expected to be a common cross-link that causes HMW species formation in mAbs. This is because therapeutic mAb products are frequently formulated in buffers with pH ranging between 5 and 6, which is also the favored condition for the reaction between primary amine and carbonyl groups to form imine bonds. Using the MS-based strategies disclosed herein, similar imine-based crosslinking chemistry in mAb covalent HMW species can be interrogated, understood and prevented.

What is claimed is:

1. A method for characterizing a covalent cross-link in a size variant of a protein of interest, comprising:
(a) subjecting a sample including a protein of interest and a size variant thereof to nSEC to form an SEC eluate;
(b) contacting said SEC eluate to a denaturing solution to form a denatured SEC eluate;
(c) subjecting said denatured SEC eluate to mass spectrometry analysis to obtain at least one mass measurement of said protein of interest and at least one mass measurement of said size variant; and
(d) comparing said mass measurements to characterize said covalent cross-link.

2. The method of claim 1, wherein said protein of interest is an antibody, a bispecific antibody, an antibody fragment, an antibody-drug conjugate, a fusion protein, or a recombinant protein.

3. The method of claim 1, wherein said denaturing solution comprises acetonitrile and formic acid.

4. The method of claim 1, wherein said denaturing solution is introduced into the SEC eluent flow using a T-mixer.

5. The method of claim 4, wherein said combined denaturing solution flow and SEC eluent flow is split into a low flow for mass spectrometry detection and a high flow for UV detection.

6. The method of claim 1, wherein said mass spectrometry analysis comprises nano-electrospray ionization.

7. The method of claim 1, wherein said covalent cross-link is selected from a group consisting of an ester bond, an amine bond, and an imine bond.

8. The method of claim 7, wherein said covalent cross-link is an imine bond.

9. A method for identifying crosslinking amino acid residues in a protein of interest, comprising:
(a) subjecting a sample including a protein of interest and a crosslinked variant thereof to chromatographic separation to form an enriched variant sample;
(b) subjecting said enriched variant sample to digestion conditions to form a peptide digest; and
(c) subjecting said peptide digest to LC-MS analysis to identify cross-linking amino acid residues.

10. The method of claim 9, wherein said crosslinked variant is a dimer.

11. The method of claim 9, wherein said chromatographic separation is selected from a group consisting of reverse phase liquid chromatography, anion exchange chromatography, cation exchange chromatography, size exclusion chromatography, affinity chromatography, hydrophobic interaction chromatography, hydrophilic interaction chromatography, and mixed-mode chromatography.

12. The method of claim 9, wherein subjecting said enriched variant sample to digestion conditions includes contacting said enriched variant sample to at least one digestive enzyme.

13. The method of claim 12, wherein said at least one digestive enzyme is selected from a group consisting of trypsin, chymotrypsin, pepsin, Tryp-N, LysN, LysC, Asp-N, Arg-C, Glu-C, and papain.

14. The method of claim 9, wherein said LC-MS analysis comprises RPLC-MS/MS analysis.

15. The method of claim 9, wherein at least one of said crosslinking amino acid residues is an oxidized variant of an amino acid residue.

16. The method of claim 9, wherein said crosslinking amino acid residues are selected from a group consisting of lysine, serine, threonine, and modified variants thereof.

17. A method for identifying a covalent cross-link in a HMW species of a therapeutic antibody, comprising:
(a) subjecting a sample including a therapeutic antibody and at least one HMW species of said therapeutic antibody to SEC to form an enriched HMW sample;
(b) subjecting said enriched HMW sample to complete or partial reduction and alkylation to form a reduced HMW sample;
(c) subjecting said reduced HMW sample to dSEC to form a very enriched HMW sample;
(d) subjecting said very enriched HMW sample to digestive conditions to form a peptide digest;
(e) subjecting said peptide digest to stabilizing conditions to form a stabilized peptide digest;
(f) subjecting said stabilized peptide digest to LC-MS/MS analysis to obtain peptide masses; and
(g) comparing said peptide masses to predicted masses of crosslinked peptides to identify said covalent cross-link.

18. The method of claim 17, wherein said partial reduction disrupts the inter-chain disulfide bonds of said therapeutic antibody.

19. The method of claim 17, wherein subjecting said enriched HMW sample to complete or partial reduction comprises contacting said enriched HMW sample to a reducing agent selected from the group consisting of dithiothreitol, β-mercaptoethanol, Ellman's reagent, hydroxylamine hydrochloride, and tris(2-carboxyethyl)phosphine hydrochloride.

20. The method of claim 17, wherein subjecting said peptide digest to stabilizing conditions comprises contacting said peptide digest to NaBH₃CN.

21. A method for selecting pH conditions for producing a therapeutic protein, comprising:
(a) subjecting a sample including a therapeutic protein and at least one crosslinked variant thereof to LC-MS analysis to determine an abundance of said at least one crosslinked variant;
(b) repeating step (a) using at least one alternative mobile phase buffered to at least one alternative pH;
(c) comparing the abundances of step (a) and step (b) to determine pH conditions at which said at least one crosslinked variant is less abundant; and
(d) using said determination to select pH conditions for producing said therapeutic protein.

22. The method of claim 21, wherein a pH of a mobile phase is about 1.5, about 2.0, about 2.5, about 3.0, about 3.5, about 4.0, about 4.5, about 5.0, about 5.5, about 6.0, about 6.5, about 7.0, about 7.5, or about 8.0.

23. The method of claim 21, wherein said at least one crosslinked variant is less abundant at an acidic pH.

24. The method of claim 21, wherein said at least one crosslinked variant is less abundant at a neutral pH.

25. The method of claim 21, wherein said at least one crosslinked variant is less abundant at a basic pH.

26. A method for identifying a covalent cross-link in a crosslinked variant of a protein of interest, comprising:
(a) contacting a sample including a crosslinked variant of a protein of interest to a first reducing agent to form a reduced sample;
(b) subjecting said reduced sample to digestion conditions to form a peptide digest;
(c) subjecting said peptide digest to LC-MS analysis to obtain a first set of peptide masses;
(d) repeating steps (a)-(c) using a second reducing agent to obtain a second set of peptide masses, wherein said second reducing agent includes a heavy isotope that is incorporated into said covalent cross-link; and
(e) comparing said first set of peptide masses and said second set of peptide masses to identify said covalent cross-link.

27. The method of claim 26, wherein said first reducing agent is NaBH₃CN and said second reducing agent is NaBD₃CN.

28. A method for detecting a covalently bound HMW species of a therapeutic antibody, comprising:
(a) subjecting a sample including a therapeutic antibody and at least one covalently bound HMW species of said therapeutic antibody to SEC to form at least one fraction including said at least one HMW species;
(b) collecting said at least one fraction to form an enriched HMW sample;
(c) subjecting said enriched HMW sample to deglycosylating conditions to form a deglycosylated sample; and
(d) subjecting said deglycosylated sample to post-column denaturation (PCD)-assisted nSEC-MS analysis to detect said covalently bound HMW species.

29. The method of claim 28, wherein subjecting said enriched HMW sample to deglycosylating conditions comprises contacting said enriched HMW sample to PNGase F.

30. A method for characterizing a covalent cross-link in a HMW species of a therapeutic antibody, comprising:
(a) subjecting a sample including a therapeutic antibody and at least one HMW species of said therapeutic antibody to SEC to form at least one fraction including said at least one HMW species;
(b) collecting said at least one fraction to form an enriched HMW sample;
(c) subjecting said enriched HMW sample to deglycosylating conditions to form a deglycosylated sample;
(d) subjecting said deglycosylated sample to digestive conditions to form a fragmented sample;
(e) subjecting said fragmented sample to complete or partial reduction and alkylation to form a reduced sample;
(f) subjecting said reduced sample to PCD-assisted nSEC-MS analysis to obtain at least one mass measurement of at least one subunit dimer of said HMW species and at least one mass measurement of each of the component subunits of said at least one subunit dimer; and
(g) comparing said mass measurements to characterize said covalent cross-link.

31. The method of claim 30, wherein subjecting said deglycosylated sample to digestive conditions comprises contacting said deglycosylated sample to IdeS or a variant thereof.

32. The method of claim 30, wherein said fragmented sample comprises antibody subunits, wherein said subunits are selected from a group consisting of Fab fragments, Fab′ fragments, Fab₂ fragments, F(ab′)₂ fragments, Fc fragments, Fc/2 fragments, Fv fragments, Fd fragments, Fd′ fragments, and combinations thereof.

33. A method for identifying crosslinking amino acid residues in a HMW species of a therapeutic antibody, comprising:
(a) characterizing a covalent cross-link in a HMW species of a therapeutic antibody according to the method of claim 30;
(b) using said characterization to predict masses of crosslinked peptides of said HMW species; and
(c) using said predicted masses to identify crosslinking amino acid residues in said HMW species according to the method of claim 17.
